(19) United States
(12) Patent Application Publication
(10) Pub. No.: US 2025/0287978 A1
(43) Pub. Date: Sep. 18, 2025

(54) Method for Producing Plant-Based Meat Analogues Using Leuconostoc Carnosum

(71) Applicant: CHR. HANSEN A/S, Hoersholm (DK)

(72) Inventors: Veronique ZULIANI, Arpajon (FR); Raquel FERNANDEZ, Hoersholm (DK); Zdenek CECH, Starovice (CZ); Cees Jan BAKKER, Nienburg/Weser (DE); Joost DE MOOIJ, Nienburg/Weser (DE)

(73) Assignee: CHR. HANSEN A/S, Hoersholm (DK)

(21) Appl. No.: 18/856,586

(22) PCT Filed: Apr. 18, 2023
(86) PCT No.: PCT/EP2023/059980

§ 371 Date: Oct. 11, 2024

(30) Foreign Application Priority Data
Apr. 20, 2022 (EP) 22169001.9

(51) Int. Cl. A23L 5/20; A23J 1/12; A23J 3/14; A23L 7/104; A23L 11/30; A23L 11/50; A23L 13/00

(52) U.S. Cl. CPC A23L 5/28; A23J 1/125; A23J 3/14; A23L 7/104; A23L 11/37; A23L 11/50; A23L 13/00

(57) ABSTRACT

The invention relates to preparation of plant-based meat analogue products with Leuconostoc carnosum. Such products can be made by inoculating a composition comprising said bacteria on plant based starting material. The invention also provides use of Leuconostoc carnosum for improving the sensory properties of such products.

### FIELD OF THE INVENTION

The present invention relates to the field of food technology and microbiology, in particular to the preparations of meat analogue products.

### BACKGROUND OF THE INVENTION

Meat alternative or meat analogues products are becoming popular in the Western world. The importance of meat alternatives continues to rise due to concerns on limited sustainability of the traditional meat production methods.

Many efforts have been made to provide different meat alternatives, in particular from plants. However, despite the good nutritional value and continuous development of plant-based meat analogues, their palatability remains a critical obstacle for consumer acceptability. For example, material prepared from legumes may have off-flavors which is less acceptable by the consumers.

The lack of animal meat flavor that consumers are familiar with and expect is a major hurdle to the progress of meat analogue products. For improving the texture and flavor of plant-based meat analogues, different ingredients are added during the manufacturing process. For example, addition of microorganisms may render the raw material more palatable, with desirable changes in the texture and organoleptic characteristics. U.S. Pat. No. 4,556,571 discloses adding yeast extract to overcome the beany flavor problem in meat alternative produces from soy.

There is an ongoing demand to provide meat analogue products with improved taste, aroma and texture.

### SUMMARY OF THE INVENTION

The present invention provides methods of producing meat analogue products from plant material, in particular from legumes or cereals, using Leuconostoc carnosum. It has been surprisingly discovered that the use of Leuconostoc carnosum leads to improved flavor profile palatability in these products, in addition to shelf-life extension.

In a first aspect, the present invention provides a process of preparing a meat analogue product comprising the steps of:
a) providing a starting material which is prepared from plant material, and
b) inoculating to the starting material with a composition comprising Leuconostoc carnosum.

Preferably, the plant material is prepared from legumes (such as soybeans, peas, beans, lupins and lentils) and/or cereal (such as oat, rice, corn and wheat).

In a second aspect, the present invention provides the use of a composition comprising Leuconostoc carnosum, preferably DSM 34220 or a mutant thereof, for improving the flavor of a meat analogue product prepared from plant material, preferably from legumes or cereals.

In a third aspect, a meat analogue product prepared from legumes and/or cereal comprising Leuconostoc carnosum, such as DSM 34220 or a mutant thereof. Preferably, there is present a concentration at least 10⁵ CFU/g Leuconostoc carnosum in the product.

### BRIEF DESCRIPTION OF THE FIGURES

FIG. 1 shows the growth of mesophilic aerobic count at Day 0, Day 8, Day 16 and Day 21 in soy-based product.

FIG. 2 shows the growth of lactic acid bacteria at Day 0, Day 8, Day 16 and Day 21 in soy-based product.

FIG. 3 shows the yeast concentration at Day 0, Day 8, Day 16 and Day 21 in soy-based product.

FIG. 4 shows the pH development at Day 0, Day 8, Day 16 and Day 21 in soy-based product.

FIG. 5 shows the cell count of artificially inoculated L. monocytogenes at Day 0, Day 10 and Day 21 in soy-based product.

FIG. 6 shows the cell count of artificially inoculated B. cereus at Day 0, Day 10 and Day 21 in soy-based product.

FIG. 7 shows the percentage of change in the level of diacetyl, pentanal, furfural, hexanal, benzaldehyde and 2-pentylfuran in the control batch and batches inoculated with Leuconostoc carnosum or Lactobacillus curvatus at Day 21, compared to control at Day 0 in soy-based product.

FIG. 8 shows the growth of mesophilic aerobic count at Day 0, Day 8, Day 16 and Day 21 in pea-based product.

FIG. 9 shows the growth of lactic acid bacteria at Day 0, Day 8, Day 16 and Day 21 in pea-based product.

FIG. 10 shows the pH development at Day 0, Day 8, Day 16 and Day 21 in pea-based product.

FIG. 11 shows the cell count of artificially inoculated L. monocytogenes at Day 0, Day 10 and Day 21 in pea-based product. The stars indicates that the concentration is above 4 Log cfu/g

FIG. 12 shows the cell count of artificially inoculated B. cereus at Day 0, Day 10 and Day 21 in pea-based product.

FIG. 13 shows the percentage of change in the level of 1-penten-3-ol pentanal, hexanal, 2-hexenal, 2-heptenal, octanal, 2-octenal, and nonanal in the control batch and batches inoculated with Leuconostoc carnosum or Lactobacillus curvatus at Day 21, compared to control at Day 0 in pea-based product.

### DETAILED DESCRIPTION OF THE INVENTION

Leuconostoc carnosum is a lactic acid bacterium that thrives in anaerobic environments with a temperature around 2° C. The slime-forming bacterium has been known to spoil vacuum-packed meat, but it is not pathogenic (Bjorkroth et al., “Identification and characterization of Leuconostoc carnosum, associated with production and spoilage of vacuum-packaged, sliced, cooked ham.” Applied and Environmental Microbiology 64.9 (1998): 3313-3319). Spoilage by Leuconostoc carnosum in meat products produces sensory changes, such as souring, gas formation, and/or slime formation.

However, the inventors of the present application have discovered novel uses of Leuconostoc carnosum for improving the flavor of meat analogue products. More specifically, the inventors have surprisingly found that Leuconostoc carnosum is able to reduce the level of off flavor compounds in meat analogue products produced from plants. In general, aldehydes are linked to the formation of off-aroma, described as beany flavor in plant-based products (Yang et al., “Sensory evaluation of oils/fats and oil/fat-based foods.” Oxidative stability and shelf life of foods containing oils and fats. AOCS Press, 2016. 157-185).

As demonstrated in the examples, treatment of the plant material with the Leuconostoc carnosum is able to reduce the aldehyde content, in particular, hexanal and pentanal. Hexanal has been reported to be the major molecule responsible for the green and herbal off-flavor perception in legume protein isolates (El Youssef et al. “Sensory improvement of a pea protein-based product using microbial co-cultures of lactic acid bacteria and yeasts.” Foods 9.3 (2020): 349).

In addition, it has been found that the volatile compound diacetyl can be reduced by Leuconostoc carnosum. Diacetyl is an important aroma compound in butter, margarine, sour cream, yogurt, and several cheeses. However, this compound gives a butter, caramel and sweet flavor which is not preferred by the consumers in meat analogue products. Moreover, diacetyl is considered as an off-odor and important contributor to spoilage in meat products (Holm, E. S., et al. “Identification of chemical markers for the sensory shelf-life of saveloy.” Meat science 90.2 (2012): 314-322).

It has been found that products treated with Leuconostoc carnosum was able to reduce furfural and benzaldehyde, which are associated with sweet and almond notes. Higher reduction in furfural and benzaldehyde can be seen compared to control.

It has furthermore been found that products treated with Leuconostoc carnosum was able to reduce the level of compounds which are, which are associated with characterized to have as beany and grassy flavor, including aldehydes (such as pentanal, hexanal, octanal and nonanal) and mono-unsaturated aldehydes (such as 2-hexenal, 2-heptenal and 2-octenal), as well as 1-penten-3-ol, which also imparts beany and green flavor.

Meat analogue products (or simply “meat analogues”) are products which are used as culinary replacements for meat products.

Examples of meat analogues include, analogues of patties, sausages, schnitzel, meat balls, meat strips, ham, steak, whole-cut meat, deli meat, burger, jerky, bacon, and the like.

It should be noted that “meat analogue” used herein does not refer to dairy or dairy analogue products, since such products are generally not considered as culinary replacements for meat.

The present invention provides a process of preparing a meat analogue product comprising the steps of a) providing a starting material which is prepared from plant material, and b) inoculating the starting material with a composition comprising Leuconostoc carnosum comprising the composition.

### Step a) Providing a Substrate

To carry out the invention, a suitable starting material is provided. This would be a starting material that comprises material prepared from plant, preferably with high content of plant proteins and/or plant fibers. Such material may be subject to chemical or enzymatic treatments or other preparation steps, to for example increase its textures or nutritional content.

Preferably, the plant material comprises legumes (such as soybeans, peas, beans, lupins and lentils) and/or cereal (such as oat, rice, corn and wheat). More preferably, the plant material comprises material prepared from soy or pea. The starting material preferably does not comprise material obtained from animal.

The term “legume” refers to any plant belonging to the family Fabaceae. Fabaceae is a large and economically important family of flowering plants, which is commonly known as the legume family, pea family, bean family or pulse family. A variety of different legumes can be consumed. Legumes typically have a pod or hull that opens along two sutures when the seeds of the legume are ripe. The Fabaceae family includes over 750 genera and 16,000 to 19,000 species.

Examples of “legumes” include peanuts (Arachis hypogaea), pigeon peas (Cajanus cajan), chickpea (Cicer arietinum), soy bean (Glycine max), lentils (Lens culinaris), lupins (Lupinus spp.), peas (Pisum sativum), field peas (Pisum arvense), beans (Phaseolus spp.), common beans (Phaseolus vulgaris) and its various cultivars and varieties, vetches (Vicia spp.), fava beans (Vicia faba), beans (Vigna spp.), cow peas (Vigna unguiculata), azuki beans (Vigna angularis) and bambara beans (Voandzeia subterranea).

The term “cereal” refers to both true cereal and pseudocereal. True cereal refers to the seeds of plants of the Poaceae family. Examples of true cereals include oat (Avena sativa), rye (Secale cereale), rice (Oryza spp. such as Oryza sativa), sorghum (Sorghum spp., such as Sorghum bicolor), triticale (Triciale), millet (such as finger millet (Eleusine coracana), foxtail millet (Setaria italica), kodo millet (Paspalum scrobiculatum), proso millet (Panicum miliaceum), barnyard millet (Echinochloa spp.)), fonio (Digitaria exilis), teff (Eragrostis tef), barley (Hordeum vulgare), corn (Zea mays), and wheat (Triticum spp.) (such as common wheat (Triticum aestivum), durum wheat (Triticum durum), club wheat (Triticum compactum), Khorasan wheat (Triticum turanicum) and spelt (Triticum spelta)).

Pseudocereal are seed of plants which do not belong to Poaceae family but are used in much the same way as cereals. Examples of pseudocereals include quinoa (Chenopodium quinoa), buckwheat (Fagopyrum esculentum), amaranth (Amaranthus tricolor), breadnut (Brosimum alicastrum), and acacia seed (Acacia spp.).

In preferred embodiments, the plant material comprises at least 10% protein, such as at least 12%, at least 15%, at least 17%, at least 20% protein, at least 25% protein.

Step b) Inoculating a Composition Comprising Leuconostoc carnosum to the Starting Material

Processes in accordance with the present invention comprise inoculating the starting material with the composition comprising Leuconostoc carnosum strain(s). It should be understood that one or more Leuconostoc carnosum strains, such as 2, 3, 4, 5 or more strains can be applied. As used herein, the term “strain” has its common meaning in the field of microbiology and refers to a genetic variant of bacterium.

Leuconostoc carnosum of the present invention may be useful for application of fermented as well as non-fermented meat analogue products.

A skilled person in the art is able to determine suitable concentrations of inoculation using routine methods and in view of the current description and examples.

In preferred embodiments, Leuconostoc carnosum is inoculated in a concentration in the range of 10⁴-10⁹ CFU/g product, e.g. in the range of 10⁴-10⁹ CFU/g product, such as in the range of 10⁵-10⁹ CFU/g product, e.g. in the range of 10⁵-10⁸ CFU/g product.

In a presently preferred embodiment, the Leuconostoc carnosum strain of the invention is added in a concentration of 10⁷ CFU/g product.

The inoculation can be carried out at a temperature of between 2-30° C. In preferred embodiments, the process further comprises fermenting the starting material.

Fermentation can be carried out at a temperature of between 2 and 25° C., such as between 2-20° C. In some preferred embodiments the fermentation temperature is between 4-15° C. In a preferred embodiment, the fermentation temperature is carried out between 20-40° C., such as 30-37° C.

Preferably, the Leuconostoc carnosum remains active in the product during the shelf life of the meat analogue products, which is preferably is at least 10 days, such as for at least 15 days, such as at least 20 days.

The composition is applied to the starting material by known methods in the art. As used herein, the term “inoculating” refers to the act of bringing the composition into contact with the starting material and allowing the bacteria to grow in the material. This could be done for example by spraying the composition, or by pouring the composition onto the material, with optional steps of mixing the composition and the starting material.

Furthermore, the composition comprising Leuconostoc carnosum and optional yeasts and/or bacteria may be in frozen, liquid or dried form, including freeze-dried form and spray/fluid bed dried form, or frozen or freeze-dried concentrates. In a preferred embodiment, the composition is freeze-dried and is diluted in water before being sprayed onto the starting material.

Leuconostoc carnosum is able to reduce off-flavor compounds in plant material, including hexanal and pentanal.

In some embodiments, hexanal is reduced by at least 10%, such at least 15%, at least 20%, at least 25%, at least 30%, at least 35%, at least 40%, at least 45%, at least 50%, at least 55%, or at least 60%.

In some embodiments, pentanal is reduced by at least 10%, such at least 15%, at least 20%, at least 25%, at least 30%, at least 35%, at least 40%, at least 45%, at least 50%, at least 55%, or at least 60%.

In some embodiments, diacetyl is reduced by at least 10%, such at least 15%, at least 20%, at least 25%, at least 30%, at least 35%, at least 40%, at least 45%, at least 50%, at least 55%, or at least 60%.

In some embodiments, benzaldehyde is reduced by at least 10%, such at least 15%, at least 20%, at least 25%, at least 30%, at least 35%, at least 40%, at least 45%, at least 50%, at least 55%, or at least 60% In some embodiments, furfural is reduced by at least 10%, such at least 15%, at least 20%, at least 25%, at least 30%, at least 35%, at least 40%, at least 45%, at least 50%, at least 55%, or at least 60%.

In some embodiments, octanal is reduced by at least 10%, such at least 15%, at least 20%, at least 25%, at least 30%, at least 35%, at least 40%, at least 45%, at least 50%, at least 55%, or at least 60%.

In some embodiments, nonanal is reduced by at least 10%, such at least 15%, at least 20%, at least 25%, at least 30%, at least 35%, at least 40%, at least 45%, at least 50%, at least 55%, or at least 60%.

In some embodiments, 2-hexenal is reduced by at least 10%, such at least 15%, at least 20%, at least 25%, at least 30%, at least 35%, at least 40%, at least 45%, at least 50%, at least 55%, or at least 60%.

In some embodiments, 2-heptenal is reduced by at least 10%, such at least 15%, at least 20%, at least 25%, at least 30%, at least 35%, at least 40%, at least 45%, at least 50%, at least 55%, or at least 60%.

In some embodiments, 2-octenal is reduced by at least 10%, such at least 15%, at least 20%, at least 25%, at least 30%, at least 35%, at least 40%, at least 45%, at least 50%, at least 55%, or at least 60%.

In some embodiments, 1-penten-3-ol is reduced by at least 10%, such at least 15%, at least 20%, at least 25%, at least 30%, at least 35%, at least 40%, at least 45%, at least 50%, at least 55%, or at least 60%.

The term “reducing” in the context of volatile compounds such as hexanal, pentanal, diacetyl, benzaldehyde and furfural refers to a lower content for a product prepared using Leuconostoc carnosum compared to the same product prepared the same way but without using Leuconostoc carnosum.

Volatile compounds described herein can be measured using known methods in the art, including gas chromatography (GC), gas chromatography-flame ionization (GC-FID), gas chromatography-mass spectrometry (GC-MS), and two-dimensional gas chromatography-mass spectrometry (GC×GC-MS).

Preferably, the Leuconostoc carnosum DSM 34220 or its mutant is used. DSM 34220 is additionally able to reduce unwanted microorganisms such as spoilage bacteria or pathogenic bacteria in the product. The term “spoilage bacteria” as used herein refers to any type of bacteria that act to spoil food. The term “food-borne pathogenic bacteria” refers to any food poisoning bacteria which can cause disease or illness in animals or humans. Examples of spoilage bacteria and pathogenic bacteria include Bacillus and Listeria, among others.

The term “mutant” should be understood as a strain derived from the Leuconostoc carnosum of the invention by means of e.g., genetic engineering, radiation and/or chemical treatment. The mutant is a functionally equivalent mutant, e.g. a mutant that has substantially the same, or improved, properties as the mother strain. In the present context, a mutant of the invention is preferable a mutant with same or improved properties with respect to flavor improvement. Such a mutant is a part of the present invention. A mutant may be a strain obtained by subjecting a strain of the invention to any conventionally used mutagenization treatment, including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 1000, no more than 100, no more than 20, no more than 10, or no more than 5, treatments are carried out. In a presently preferred mutant, less than 5%, or less than 1% or even less than 0.1% of the nucleotides in the bacterial genome have been changed (such as by replacement, insertion, deletion or a combination thereof) compared to the mother strain. The functionally equivalent mutant may reduce hexanal or pentanal when compared to the mother strain tested under the same condition.

Use of other Leuconostoc carnosum strains are also within the scope of the present application.

The composition used in the present application, in addition to Leuconostoc carnosum such as deposited a DSM 34220, further comprises other yeast(s) (such as Debaryomyces hansenii or Pichia kluyveri) and/or other bacteria (such as Pediococcus, Lactococcus and Staphylococcus which is preferably coagulase-negative). In some preferred embodiments, the bacteria is lactic acid bacteria or Lactobacillus spp.

In preferred embodiments, the composition further comprises Lactococcus lactis, Lactobacillus sakei, Lactobacillus curvatus, Pediococcus acidilactici, Pediococcus pentocaseus, Staphylococcus carnosus, Staphylococcus xylosus, and/or Staphylococcus vitulinus.

More preferably, the composition further comprises Lactococcus lactis deposited as DSM 11037, Lactobacillus sakei deposited as DSM 14022, Lactobacillus curvatus deposited as DSM 18775, Pediococcus acidilactici deposited as DSM 28307, Staphylococcus carnosus DSM 25010 or DSM 32779, Staphylococcus xylosus DSM 28308 and/or Staphylococcus vitulinus DSM 25789.

In other embodiments, the composition comprises Leuconostoc carnosum only, so that Leuconostoc carnosum is the only bacteria which is applied to the starting material.

The plant material which is treated with Leuconostoc carnosum may be subjected to further processing steps, such as fermentation with additional microorganisms as starter culture. An additional fermentation step may be carried out. During the preparation process, a skilled person in the art is able to adjust other parameters known to him in order to achieve the desired end-product.

In other aspects, the present invention also provides meat analogue product obtained by the processes described herein. The meat analogue products comprise Leuconostoc carnosum, such as DSM 34220, and optionally, further yeasts and/or bacteria as described herein.

In further aspects, the present invention provides meat analogue products comprising Leuconostoc carnosum, preferably in a concentration of at least 10⁵ CFU/g, such as at least at least 10⁶ CFU/g, at least 10⁷ CFU/g, at least 10⁸ CFU/g or higher.

The present invention is especially useful for preparing meat analogue products from material of plant origin. Included herein are legumes (such as soybeans) and/or cereals (oat, rice, corn, or wheat). Preferred legumes include soybeans, peas, beans, lupins, lentils.

Meat analogues prepared from soy has been known for some time. However, it is always faced with the challenge of off flavor such as beany flavor, something not familiar to the Western consumers and is a barrier for consumption.

An important aspect of the present applicant is the use of Leuconostoc carnosum for improving the flavor of meat analogue products. The term “improving the flavor” of a product refers to making the product more palatable, compared to a product produced the same way but without inoculating with Leuconostoc. The assessment can be made by sensory evaluation using techniques known in the art, such as descriptive analysis by trained panelists (Lawless et al., “Sensory Evaluation of Food: Practices and Principals.” Food Science Texts Series. Chapman and Hall, New York (2010)). The assessment can also be made by volatile organic compound (VOC) analysis of flavor compounds described herein.

According to the present invention, Leuconostoc carnosum can be used to reduce the level of some volatile compounds which are undesirable in plant-based meat alternative products. It has been found that the level of hexanal, pentanal, diacetyl, benzaldehyde and furfural are reduced.

Accordingly, the present invention provides the uses of Leuconostoc carnosum to reduce hexanal, pentanal, diacetyl, benzaldehyde and/or furfural content in a meat analogue product prepared from plant material such as legumes or cereal. In preferred embodiments, the uses involve inoculating to the plant based starting material with a composition comprising Leuconostoc carnosum and allowing the bacteria to grow in the material.

The present invention makes use of a composition comprising a Leuconostoc carnosum, which can optionally further comprise additional yeasts and/or bacteria strain(s). The composition is preferably a high-density culture, more preferably in a frozen, dried or freeze-dried form. However, the composition may also be a liquid that is obtained after suspension of the frozen, dried or freeze-dried cell concentrates in a liquid medium such as water or PBS buffer.

Where the composition of the invention is a suspension, the concentration of viable cells is in the range of 10⁷ to 10¹⁰ cfu (colony forming units) per ml of the composition, including at least 10⁷ cfu per ml of the composition, such as at least 10⁸ cfu/ml, e.g. at least 10⁹ cfu/ml, such as at least 10¹⁰ cfu/ml.

In a preferred embodiment, the composition which is a high-density culture may have a concentration of viable cells of at least 10⁹ CFU/g colony forming units (CFU)/g, such as at least 10¹⁰ CFU/g, such as at least 1011 CFU/g, such as at least 10¹² CFU/g, such as at least 10¹³ CFU/g.

The composition of the present invention may additionally comprise cryoprotectants, lyoprotectants, antioxidants, nutrients, fillers, flavorants or mixtures thereof. The composition may be in frozen or freeze-dried form. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In further aspects, the present application provides plant-based meat analogue products comprising Leuconostoc carnosum, in particular DSM 34220. Preferably, such products are analogues of patties, sausages, schnitzel, meat balls, meat strips, ham, steak, whole-cut meat, deli meat, burger, jerky, bacon, made from legume and/or cereal.

The manufacturing of meat alternatives aims to create a meat-like structure, a meat-like appearance, create a meat-like flavor. The characteristic and dominant feature of consumable meat is its fibrous structure and texture.

In preferred embodiments, the plant material is structured from plant proteins to mimic the consistency of meat product. Such processes are known in the art and for example have described in Dekkers, “Structuring processes for meat analogues.” Trends in Food Science & Technology 81 (2018): 25-36. Known methods include extrusion, sheer cell and fiber spinning.

Extrusion is a well-developed technology in the food industry, first designed to manufacture pasta products during the 1930s. This process involves the transformation and molding of food mixtures by driving them through a die, applying heat and pressure, and using a mechanical shear to obtain the desired sizing. A typical extrusion process can be divided into three steps, that is, the initial preparation of the food material before the addition into the extruder, the ingredients are then cooked and mixed together to obtain a homogeneous texture within the barrel of the extruder, and finally the resulting product is left to cool to maintain its final shape.

Shear cell technology is a more energy-efficient structuring process that was more recently introduced. This procedure was inspired by the effect of shear flow on dough and is effective for producing meat analogues when functioning at raised temperatures. Shear-induced structuring can be achieved with shear cell. Depending on the processing conditions, fibrous, layered, or homogeneous samples can be obtained.

The fiber spinning techniques were adopted from the spun fiber method to create synthetic fibers in the textile industry. Fibers are made by creating filaments out of the protein used as the starting material. The process begins through the dispersion of proteins into a dispersing medium such as an alkaline aqueous solution. This dispersion is then fed through a spinneret, a device used to extrude a polymer solution to form fibers and deposited into an acidic salt solution. After exiting the spinneret's small die, the filaments would be stretched and elongated until the average thickness is about 20 microns. The excess salt solution is then removed from the fibers through squeezing or centrifuging before further processing. After the drying process, edible binders are added to keep the fibers physically tied together through functioning as an adhesive or serving as a matrix in which the fibers embedded. The fibers are then passed through a bath of melted fat and pressed together, and then cut into a suitable length.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

TABLE 1
Deposits made at a Depositary institution having acquired the
status of international depositary authority under the Budapest
Treaty on the International Recognition of the Deposit of
Microorganisms for the Purposes of Patent Procedure: Leibniz
Institute DSMZ-German Collection of Microorganisms and
Cell Cultures Inhoffenstr. 7B, 38124 Braunschweig, Germany.

| Strain | Accession No. | Deposit date |
|---|---|---|
| Lactococcus lactis | DSM 11037 | 1996 Jun. 26 |
| Lactobacillus sakei | DSM 14022 | 2001 Jan. 31 |
| Lactobacillus curvatus | DSM 18775 | 2006 Nov. 9 |
| Staphylococcus carnosus | DSM 25010 | 2011 Jul. 12 |
| Staphylococcus vitulinus | DSM 25789 | 2012 Mar. 15 |
| Pediococcus acidilactici | DSM 28307 | 2014 Jan. 30 |
| Staphylococcus xylosus | DSM 28308 | 2014 Jan. 30 |
| Staphylococcus carnosus | DSM 32779 | 2018 Mar. 20 |
| Leuconostoc carnosum | DSM 34220 | 2022 Mar. 8 |

### Examples

Plant-based meat analogue are sensitive products with short shelf life even at chilled temperature. Moreover, they can be contaminated with pathogenic bacteria such as Listeria monocytogenes and Bacillus cereus. They can also have beany unpleasant off flavor.

The following examples demonstrate the advantages of Leuconostoc carnosum to improve the overall quality of plant based minced meat analogue made from soy or pea, compared with Lactobacillus curvatus (a bacterial culture used in meat products as described in EP2132297B).

### A. Soy-Based Product

### A.1. Material and Methods

### A.1.1 Starting Material

Freshly produced minced meat analogue prepared from soy (containing water, soy protein concentrate, coconut oil, rapeseed oil, methylcellulose, soy protein isolate, vinegar, starch, aroma, salt, coloring) was used as starting material.

### A.1.2 Preparation

3 batches were prepared from the starting material as follows:
Batch 1—control without inoculation of bacterial culture
Batch 2—inoculated with a bacterial culture of Leuconostoc carnosum DSM 34220
Batch 3—inoculated with a bacterial culture Lactobacillus curvatus DSM 18775.

Bacterial cultures were provided in freeze dried form and diluted in cold tap water (<12° C.) and mixed to obtain a homogenous bacterial suspension. The suspension was then added at a ratio of 1% (v/w) into the starting material and mixed to have a homogeneous distribution of the bacteria within the starting material. The range of inoculation was between 6.7 and 7.0 Log cfu/g.

After proper mixing, samples were packed and stored for 21 days at 7° C. under modified atmosphere conditions (70% N2 and 30% CO₂) for shelf-life study, challenge test, and volatile organic compound (VOC analysis).

### A.1.3.1 Shelf-Life Study

Samples from Day 0, Day 8, Day 16 and Day 21, were taken from batches 1-3 and analyzed for the following microorganisms using ISO international enumeration standards (3 replicates):
lactic acid bacteria (LAB, ISO 15214:1998),
mesophilic aerobic count (MAC, ISO 4833-1:2013), and
yeast and mold (ISO 21527-1:2008).

MAC was enumerated to give an overall estimation of the bacterial flora present in the product. LAB was enumerated to evaluate the implementation at Day 0 and the subsequent growth of the food culture. LAB count can be also compared with MAC to evaluate if LAB is the dominating flora of the product in the different Batches. Yeasts and molds are known to be major spoilers of plant based products.

A.1.3.2 Challenge Test of Listeria monocytogenes and Bacillus cereus

At Day 0, additional samples from batch 1, 2 and 3 were inoculated with a cocktail of 2 Listeria monocytogenes strains or a cocktail of 2 Bacillus cereus strains in a cold and starvation stress physiological stage to get an initial bacterial concentration close to 2.0 Log cfu/g.

At Day 0, Day 10 and Day 21, Listeria or Bacillus cell count as well as some physico-chemical parameters were measured using indicated methods:
Listeria monocytogenes (BRD 07/17-01/09) or Bacillus level (ISO 7932:2004) (3 replicates)
pH (1 replicate/Day 0, 10, and 21, NF V04-408), and
water activity (1 replicate at Day 0, ISO 21807:2005).

### A.1.4 Volatile Organic Compound (VOC) Analysis

Samples of batches 1-3 from Day 0 and Day 21 were further cooked prior to VOC measurement, using the following steps:
1. kneading the sample without opening the pouch to avoid contaminations for homogenization
2. filling 400 g of the sample evenly into shape in a foil pan
3. putting a thermometer into the core of the sample
4. placing the foil pan with the sample on the furnace grate (furnace setup: 160° C., air circulation)
5. baking until core temperature of 90° C. was reached, and
6. cutting the final product into approx. 1.5 cm thick slices and dispensing 3 grams of sample into a 20 ml headspace vial for VOC analysis. Vials were capped tightly and stored at −18° C. until analysis. Samples were prepared in triplicates.

Afterwards, volatile organic compounds (VOC) were analyzed by head space solid phase microextraction gas chromatography coupled to mass spectrometry (HS-SPME-GC-MS). The instrument was a Multi-Purpose Sampler (Gerstel, MSCI, Skovlunde, Denmark), with a 7890B GC (Agilent Technologies, Denmark) and a 5977A MS (Agilent Technologies, Denmark). VOCs were extracted by SPME using a DVB/Car/PDMS-fiber (Supelco #57299, VWR, Denmark) for 20 min. at 60° C., desorbed splitless at 270° C. onto a TenaxTA-filled liner (Gerstel #012438, MSCI, Skovlunde, Denmark) kept at −30° C. After fiber desorption, the TenaxTA-filled liner were heated to 300° C. and the trapped VOCs transferred splitless and separated on a DB-5MS UI column 30 m×0.25 mm×1 μm (Agilent #122-5533UI, Agilent Technologies, Denmark) at 170 kPa constant pressure using helium as carrier gas. Oven temperature program was as follows: starting at 32° C./2 min—increased to 102° C.@10° C./min—further increased to 145° C.@5° C./min—further increased to 200° C.@15° C./min—further increased to 200° C.@15° C./min—further increased to 280° C.@20° C./min—hold at 280° C. for 5 min. The mass spectrometer operated in electron impact mode at −70 eV and the analyzer was scanning from 29-209 amu.

NIST 17 library search and Retention Indexes were used for identification of VOCs. Feature extraction was done using MassHunter Quantitative Analysis (Version 10.1, Build 10.1.733.0, Agilent Technologies, Denmark) and results calculated as peak height divided by baseline noise (signal-to-noise).

### A.2 Results

### A.2.1 Shelf-Life Study

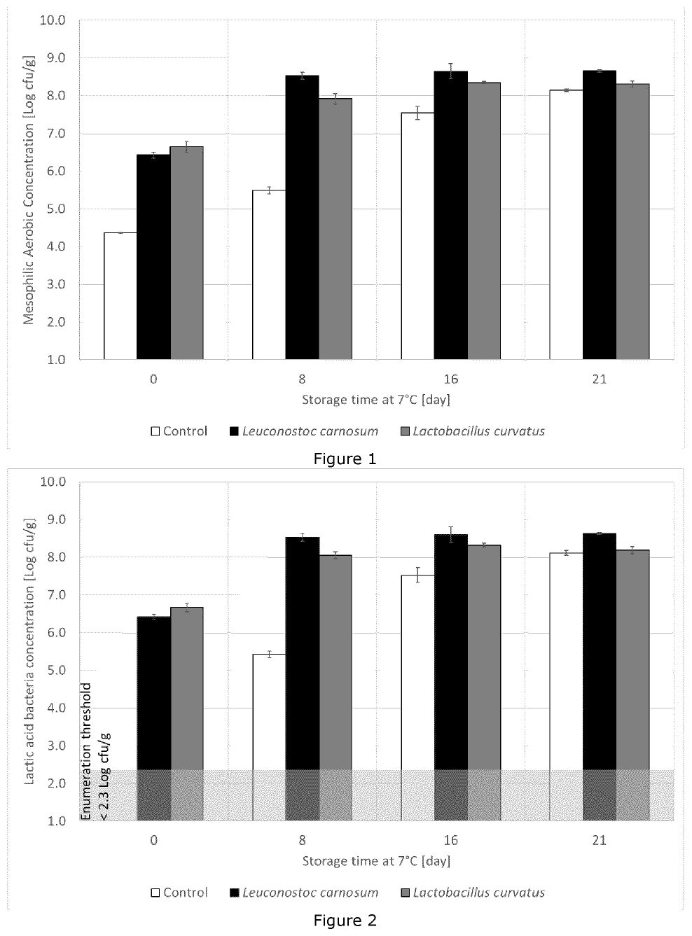

At Day 0 the mesophilic aerobic count (MAC) level was lower in the control sample compared to the samples in which bacterial cultures were added (FIG. 1). The addition of cultures increased indeed MAC level from Day 0. In all batches, the level of MAC increased until reaching a maximal concentration between 8 and 8.5 Log cfu/g.

This shows that the cultures tested were able to grow in plant-based products. The endogenous flora naturally present in such a kind of products were also able to grow significantly.

One can observe that LAB was the dominating flora in the samples containing the cultures from Day 0 and until Day 21 (FIGS. 1 and 2). The added cultures can indeed grow on both agar media used to enumerate MAC and LAB. In contrast, the LAB flora was not the dominating flora at Day 0 in the control batch but became the dominating population from Day 8 (FIG. 2).

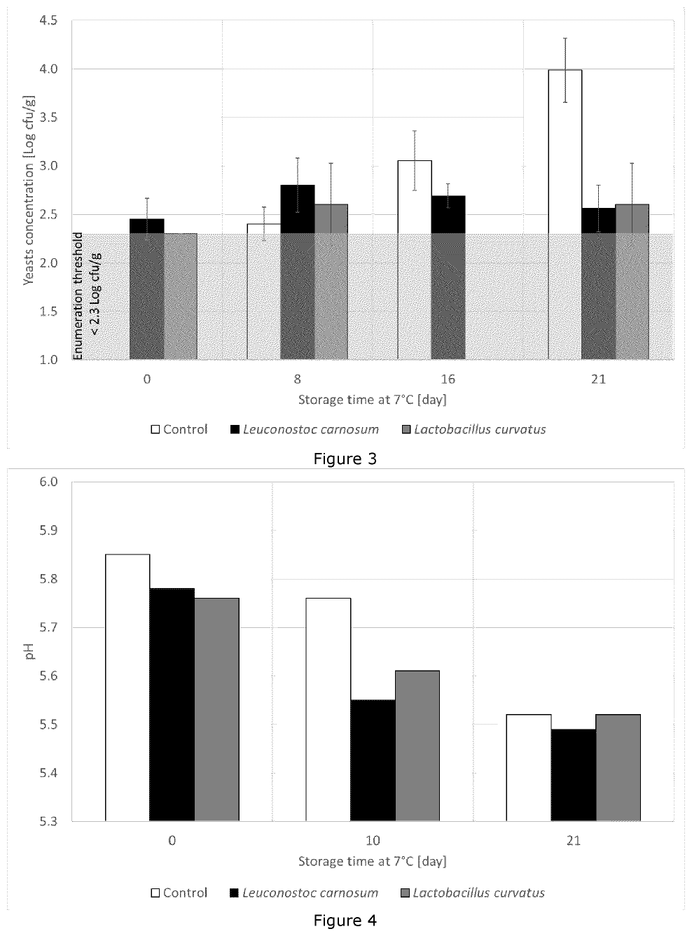

The cultures can significantly delay the growth of endogenous yeasts and molds (FIG. 3). While the concentration was below 2.5 Log cfu/g in all samples at Day 0, a significant growth takes place in the control sample (up to 4.0 Log cfu/g at Day 21) while it doesn't exceed 2.6 Log cfu/g in the samples with culture.

### A.2.2 Challenge Tests Study

### A.2.2.1 Physico-Chemical Results

pH

The pH of the samples is shown in FIG. 4. At the beginning of the study, pH was around 5.8. A slight drop in pH was observed (0.3-0.4 pH unit) between Day 0 and Day 21. The final pH was not significantly impacted by the addition of any of the bacterial cultures but the acidification was faster when they were added.

### Water Activity

The water activity was very high and not influenced by the culture addition. It was close to 0.99 and thus suitable for the growth of microorganisms in general during the whole shelf life of plant-based products.

### A.2.2.2 Challenge Test Results

Listeria monocytogenes

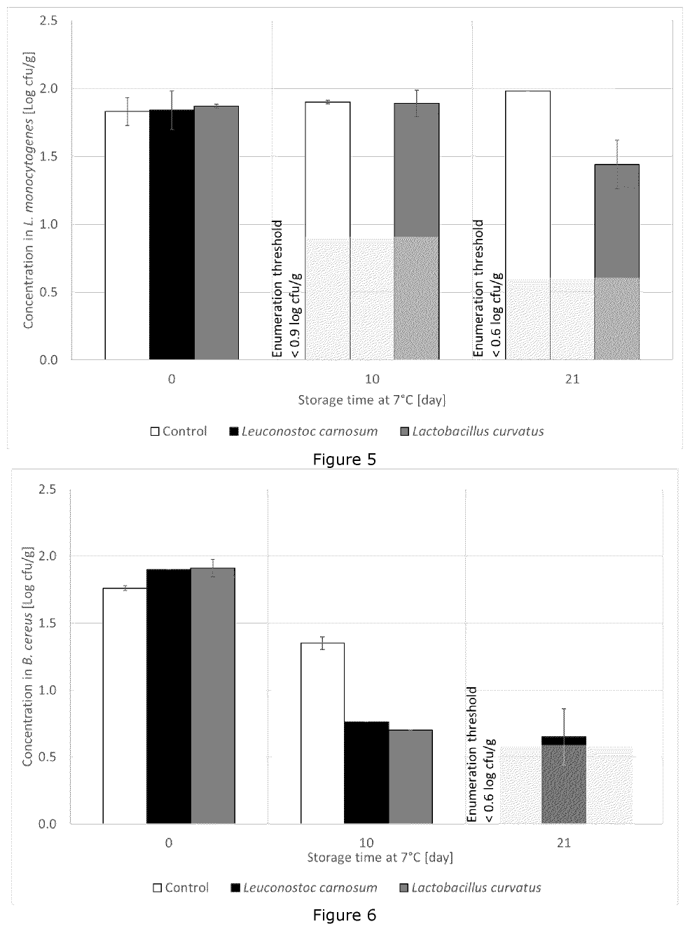

During the 21 days of the study, a stable L. monocytogenes count was measured for the control batch. However, a drop of 0.4 Log cfu/g was observed with the batch inoculated with Lactobacillus curvatus (FIG. 5). Advantageously, the decrease of the pathogenic population was even faster using Leuconostoc carnosum: the concentration dropped from 1.8 to below 0.9 Log cfu/g in the first 10 days. At day 21, the concentration in Listeria monocytogenes decreased again below the enumeration threshold: 0.6 Log cfu/g at that time, meaning that compared to the concentration at Day 0, the Listeria population was at least reduced by 1.24 Log cfu/g.

Bacillus cereus

The Bacillus cereus concentration drops in the 3 tested batches from around 1.8 to close or below 0.6 Log cfu/g (enumeration threshold) after 21 days of shelf life at 7° C. (FIG. 6). The decrease was nevertheless significantly faster in the batches inoculated with cultures. Indeed, at Day 10, while the B. cereus concentration was at 1.4 Log cfu/g in the control, the concentrations were already reduced to 0.8 and 0.7 Log cfu/g respectively for the samples with Leuconostoc carnosum and Lactobacillus curvatus.

### A.2.3 VOC Analysis Results

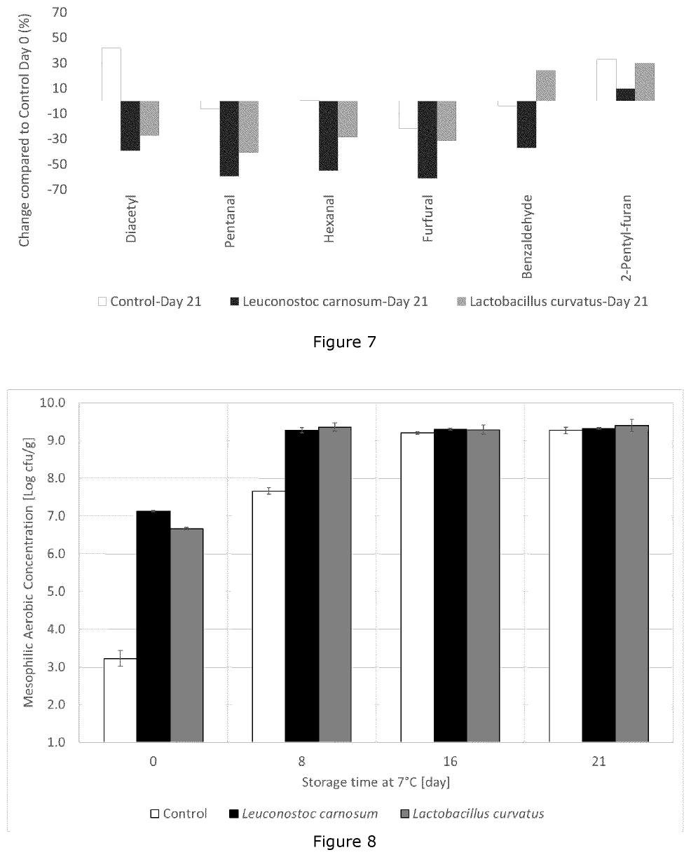

Key volatile flavor metabolites were measured as described in section A.1.4. Control batch at Day 0 and Day 21 were compared to batches inoculated with Leuconostoc carnosum or Lactobacillus curvatus. Volatile compounds showing differences above the Limit of Quantification (which is typically determined by a signal-to-noise value of 10) are shown in Table 2. FIG. 7 shows the reduction of the compound level compared to the control at day 21.

TABLE 2

| | Response (Signal to noise) | | | |
|---|---|---|---|---|
| | | | Lacto- | Leuco- |
| | | | bacillus | nostoc |
| | Control | Control | curvatus | carnosum |
| Compound | day 0 | day 21 | day 21 | day 21 |
| Pentanal | 458 ± 41 | 426 ± 55 | 281 ± 20 | 185 ± 33 |
| Hexanal | 4370 ± 393 | 4336 ± 434 | 3113 ± 187 | 1953 ± 234 |
| 2- | 5782 ± 174 | 7683 ± 1306 | 7677 ± 384 | 6341 ± 254 |
| Pentylfuran | | | | |
| Diacetyl | 1691 ± 507 | 2290 ± 206 | 1582 ± 158 | 958 ± 77 |
| Benzal- | 6127 ± 368 | 5911 ± 1182 | 7105 ± 284 | 3840 ± 538 |
| dehyde | | | | |
| Furfural | 48 ± 6 | 37 ± 6 | 32 ± 2 | 19 ± 2 |

In Table 2, the results are represented as the average of triplicates (in signal-to-noise) and the uncertainty on the measurements is calculated based on the standard deviation of those triplicates.

### Aldehydes

In general, aldehydes are linked to the formation of off flavor. It is often described as beany flavor in soy-based products. As shown, pentanal and hexanal, two key aldehydes which give green and beany off-flavor, were advantageously reduced (see Table 2, FIG. 7).

The results show that product inoculated with Leuconostoc carnosum after 21 days has the lowest aldehyde content, based on those two compounds, compared to control as well as the product inoculated with Lactococcus curvatus.

### 2-Pentyl Furan

The furan-2-pentyl furan—was known to be responsible for the beany, grassy flavor of oxidized soybean oil (Chang et al. “Isolation and identification of 2-pentyl-furan as contributing to the reversion flavor of soyabean oil.” Chemistry & Industry 46 (1966): 1926-1927) and also one of the molecules leading to an off-flavor in soy products (Min et al. “Effect of soybean varieties and growing locations on the flavor of soymilk.” Journal of Food Science 70.1 (2005): C1-C11). 2-Pentyl furan increased in the control at day 21. In contrast, the compound is less present if the product is inoculated with Leuconostoc carnosum.

### Diacetyl

Diacetyl is considered as an off-odor in meat products and important contributor to spoilage (Holm, E. S., et al. “Identification of chemical markers for the sensory shelf-life of saveloy.” Meat science 90.2 (2012): 314-322). It imparts a butter, caramel and sweet flavor. In the present application, it was observed that diacetyl increased in the control. However, treatment with Leuconostoc carnosum leads to the degradation of the off-flavor compound.

### Furfural and Benzaldehyde

Furfural is a product of the Maillard reaction and contributes sweet, almond, and bread odors to products (Bi et al. “Characterization of key aroma compounds in raw and roasted peas (Pisum sativum L.) by application of instrumental and sensory techniques.” Journal of agricultural and food chemistry 68.9 (2020): 2718-2727). Furfural has been reported as a soy sauce-like flavor (Xu et al. “HS-SPME-GC-MS/olfactometry combined with chemometrics to assess the impact of germination on flavor attributes of chickpea, lentil, and yellow pea flours.” Food Chemistry 280 (2019): 83-95). Benzaldehyde was identified as aroma-active compounds in raw pea samples which gives an overall almond scented aroma (Bi et al., 2020). It imparts a strong, sharp, sweet, bitter almond and cherry flavor. Both compounds are not desired in meat analogue products due to their sweet and almond flavor contributions.

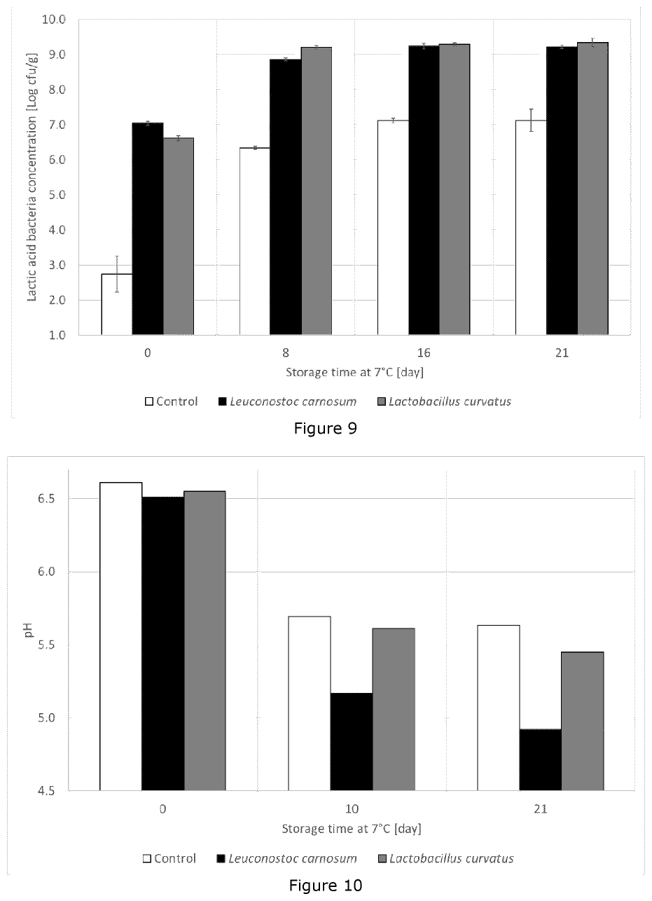

The results show that furfural and benzaldehyde can be decreased to a larger extent when Leuconostoc carnosum was applied (FIG. 9).

In sum, the results show that the use of Leuconostoc carnosum in meat analogue products can reduce naturally occurring off-flavor such as beany flavor in legumes material, in addition to shelf-life extension, which results in a more appealing plant-based product.

### B. Pea-Based Product

### B.1. Material and Methods

### B.1.1 Starting Material

Freshly produced minced meat analogue was prepared from pea using the following method: First the textured vegetable pea protein was diluted into water and blended with colorings. The dough was then grinded (5 mm plate) and blended again with additional water, ice and other ingredients (except fibers and coconut fat).

Coconut fat was grinded separately (3 mm plate) before being incorporated into the dough. Fibers were the last ingredient added and blended until proper homogenization.

The end material contained water, textured pea protein, coconut oil, sunflower oil, pea protein, citrus fiber, potassium lactate, tomato puree, methyl cellulose, colorings, white pepper, black pepper, onion powder, mace, nutmeg salt, sunflower lecithin, yeast extract.

### B.1.2 Preparation

3 batches were prepared from the starting material as follows:
Batch 1—control without inoculation of bacterial culture
Batch 2—inoculated with a bacterial culture of Leuconostoc carnosum DSM 34220
Batch 3—inoculated with a bacterial culture Lactobacillus curvatus DSM 18775.

Bacterial cultures were provided in freeze dried form and diluted in cold tap water (<12° C.) and mixed to obtain a homogenous bacterial suspension. The suspension was then added at a ratio of 1% (v/w) into the starting material and mixed to have a homogeneous distribution of the bacteria within the starting material. The range of inoculation was between 6.7 and 7.1 Log cfu/g.

After proper mixing, samples were packed and stored for 21 days at 7° C. under modified atmosphere conditions (70% N2 and 30% CO₂) for shelf-life study, challenge test, and volatile organic compound (VOC analysis).

### B.1.3.1 Shelf-Life Study

Samples from Day 0, Day 8, Day 16 and Day 21, were taken from batches 1-3 and analyzed for the following microorganisms using ISO international enumeration standards (3 replicates):
lactic acid bacteria (LAB, ISO 15214:1998),
mesophilic aerobic count (MAC, ISO 4833-1:2013)
yeast and mold (ISO 21527-1:2008).

MAC was enumerated to give an overall estimation of the bacterial flora present in the product. LAB was enumerated to evaluate the implementation at Day 0 and the subsequent growth of the food culture. LAB count can be also compared with MAC to evaluate if LAB is the dominating flora of the product in the different Batches.

B.1.3.2 Challenge Test of Listeria monocytogenes and Bacillus cereus

At Day 0, additional samples from batch 1, 2 and 3 were inoculated with a cocktail of 2 Listeria monocytogenes strains or a cocktail of 2 Bacillus cereus strains in a cold and starvation stress physiological stage to get an initial bacterial concentration close to 2.0 Log cfu/g.

At Day 0, Day 10 and Day 21, Listeria or Bacillus cell count as well as some physico-chemical parameters were measured using indicated methods:
Listeria monocytogenes (BRD 07/17-01/09) or Bacillus level (ISO 7932:2004) (3 replicates)
pH (1 replicate/Day 0, 10, and 21, NF V04-408), and
water activity (1 replicate at Day 0, ISO 21807:2005)

### B.1.4 Volatile Organic Compound (VOC) Analysis

Samples of batches 1-3 from Day 0 and Day 21 were further cooked prior to VOC measurement as described above in section A.1.4.

### B.2 Results

### B.2.1 Shelf-Life Study

At Day 0 the MAC level was lower in the control sample compared to the samples in which bacterial cultures were added (FIG. 8). The addition of cultures increased indeed MAC level from Day 0. In all batches, the level of MAC increased until reaching a maximal concentration between 9 and 9.5 Log cfu/g.

This shows that the cultures tested were able to grow in pea-based products. The endogenous flora naturally present in such kind of products were also able to grow significantly.

One can observe that lactic acid bacteria (LAB) was the dominating flora in the samples containing the cultures from Day 0 and until Day 21 (FIG. 9). In contrast, the LAB was not the dominating flora at Day 0 in the control, and despite strong growth (+4.4 Log cfu/g in 21 days), it was still not the dominating flora at D21 (FIG. 9).

The yeast and mold concentration was below the enumeration threshold (2.3 Log cfu/g) for all samples during the shelf life study (Figure not shown).

### B.2.2 Challenge Tests Study

### B.2.2.1 Physico-Chemical Results

The pH of the samples in shown in FIG. 10. At the beginning of the study, pH was around 6.6. A significant drop in pH was observed (1.0-1.6 pH unit) between DO and D21.

While the final pH is not significantly different between the control and the batch inoculated with Lactobacillus curvatus. It is significantly lower when Leuconostoc carnosum was added.

### Water Activity

The water activity was very high and not influenced by the culture addition. It was close to 0.98-0.99 and thus suitable for the growth of microorganisms in general during the whole shelf life of plant-based products.

### B.2.2.2 Challenge Test Results

Listeria monocytogenes

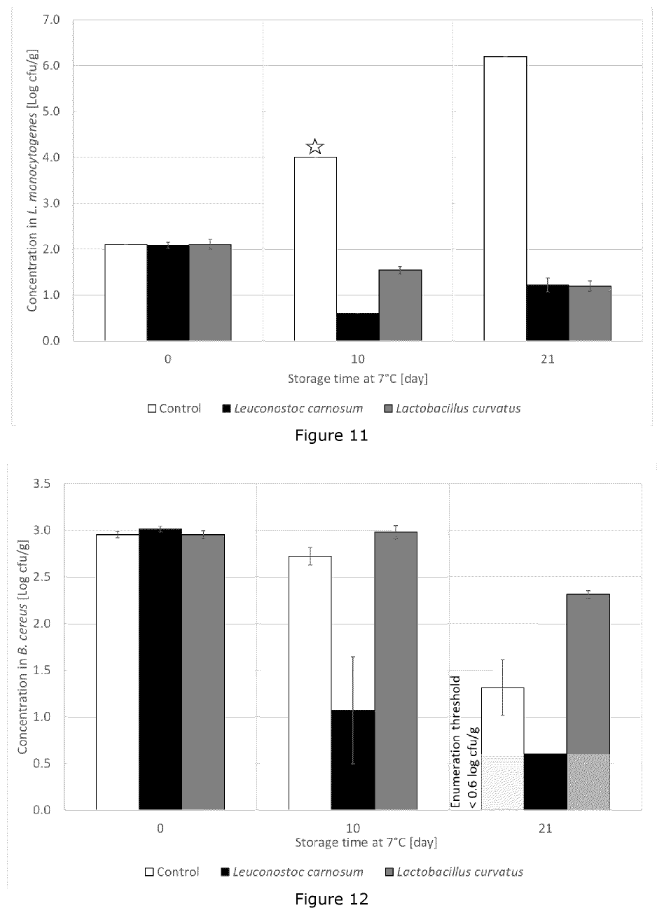

During the 21 days of the study, a significant growth of L. monocytogenes count was measured for the control batch (+4.1 Log cfu/g). However, a drop of 0.6 Log cfu/g was observed with the batch inoculated with Lactobacillus curvatus (FIG. 11).

Advantageously, the decrease of the pathogenic population was even faster when Leuconostoc carnosum was applied: the concentration dropped from 2.1 to 0.6 Log cfu/g in the first 10 days.

At day 21, the concentration of Listeria monocytogenes compared to the concentration at Day 0 dropped at least by 0.9 Log cfu/g when both tested cultures were applied.

Bacillus cereus

The Bacillus cereus concentration measured at day 0 was 3.0 cfu/g, which was higher than expected. The Bacillus cereus concentration dropped in the 3 tested batches from around 3.0 to below 0.6 Log cfu/g (enumeration threshold) after 21 days of shelf life at 7° C. (FIG. 12) in the batch inoculated with Leuconostoc carnosum. The decrease was nevertheless significantly slower and less strong in the batches inoculated with Lactobacillus curvatus and in the control batch. Indeed, at Day 21, the B. cereus concentration was at 1.3 Log cfu/g in the control, and at 2.3 in the batch with Lactobacillus curvatus.

### B.2.3 VOC Analysis Results

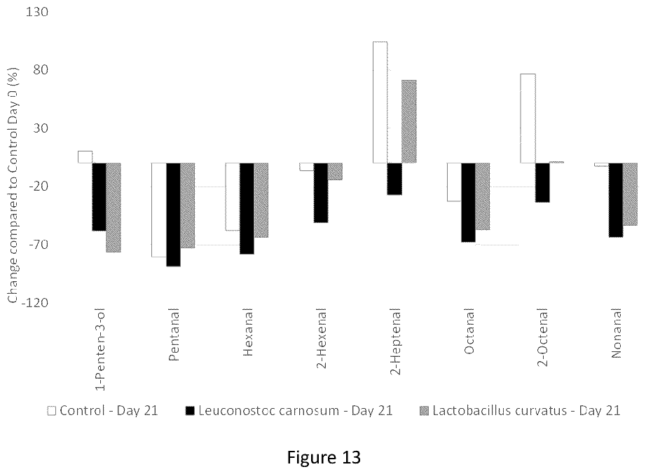

Key volatile flavor metabolites were measured as described in section A.1.4. Control batch at Day 0 and Day 21 were compared to batches inoculated with Leuconostoc carnosum or Lactobacillus curvatus at day 21. Volatile compounds showing differences above the Limit of Quantification (which is typically determined by a signal-to-noise value of 10) are shown in Table 3. FIG. 13 shows the reduction of the compound level compared to the control at day 21.

TABLE 3

| | Response (Signal to noise) | | | |
|---|---|---|---|---|
| | | | Lactobacillus | Leuconostoc |
| | Control | Control | curvatus | carnosum |
| Compound | day 0 | day 21 | day 21 | day 21 |
| Pentanal | 933 ± 6 | 180 ± 10 | 253 ± 40 | 105 ± 24 |
| Hexanal | 1139 ± 50 | 479 ± 39 | 417 ± 33 | 251 ± 36 |
| Octanal | 416 ± 42 | 280 ± 47 | 177 ± 23 | 133 ± 6 |
| Nonanal | 123 ± 18 | 120 ± 12 | 57 ± 5 | 45 ± 2 |
| 2-Hexenal | 32 ± 5 | 30 ± 3 | 27 ± 2 | 16 ± 2 |
| 2-Heptenal | 101 ± 13 | 206 ± 18 | 172 ± 35 | 73 ± 13 |
| 2-Octenal | 65 ± 10 | 115 ± 15 | 65 ± 13 | 43 ± 3 |
| 1-Penten-3-ol | 51 ± 3 | 56 ± 4 | 12 ± 7 | 21 ± 8 |

In Table 3, the results are represented as the average of triplicates (in signal-to-noise) and the uncertainty on the measurements is calculated based on the standard deviation of those triplicates.

### Aldehydes

Saturated aldehydes come from the degradation of fatty acids and are associated with green, grassy and vegetable notes in pea-based products (Xiang et al., Volatile compounds analysis and biodegradation strategy of beany flavor in pea protein, Food Chemistry 402, 134275 (2023).

Pentanal, hexanal, octanal and nonanal were significantly decreased (see Table 3, FIG. 13). Those compounds have been associated with beany and grassy aromas in plant proteins (Engels et al., “Metabolic Conversions by Lactic acid bacteria during Plant Protein Fermentations”. Foods 11, 1005 (2022)).

A decrease can also be observed for mono-unsaturated aldehydes such as 2-hexenal, 2-heptenal and 2-octenal, which have been described as green and grass in pea protein-bases (Youseff et al. Sensory Improvement of a Pea Protein-Based Product Using Microbial Co-Cultures of Lactic Acid Bacteria and Yeasts, Foods 9, 349 (2022)). 2-octenal was included in the list of key-odorants as relevant for the aroma of pea preparations (Trindler et al., Aroma of peas, its constituents and reduction strategies—Effects from breeding to processing, Food Chemistry 376, 131892 (2022)).

As previously seen in soy, the results here demonstrate that product inoculated with Leuconostoc carnosum after 21 days has the lowest aldehyde content, based on those compounds, compared to control as well as the product inoculated with Lactococcus curvatus in pea-based products.

### 1-Penten-3-ol

The alcohol 1-penten-3-ol possesses beany and green flavor according to Xu et al. “HS-SPME-GC-MS/olfactometry combined with chemometrics to assess the impact of germination on flavor attributes of chickpea, lentil, and yellow pea flours, Food Chemistry 280, 83-9584 (2019); Youseff et al. Sensory Improvement of a Pea Protein-Based Product Using Microbial Co-Cultures of Lactic Acid Bacteria and Yeasts, Foods 9, 349 (2022)).

1-Penten-3-ol increased in the control at day 21. In contrast, the compound is degraded when the product was inoculated with Leuconostoc carnosum.

In sum, the results show that the use of Leuconostoc carnosum in meat analogue products based on pea can reduce naturally occurring off-flavor such as beany and green flavor, in addition to shelf-life extension, which results in a more appealing plant-based product.

1. A process of preparing a meat analogue product comprising the steps of:
a) providing a starting material which is prepared from plant material, and
b) inoculating to the starting material with a composition comprising Leuconostoc carnosum, preferably Leuconostoc carnosum deposited as DSM 34220 or a mutant thereof.

2. The process of claim 1, wherein the plant material is prepared from legumes and/or cereal.

3. The process according to any one of the preceding claims, further comprising mixing the starting material with the composition.

4. The process according to any one of the preceding claims, further comprising fermenting the starting material.

5. The process according to claim 4, wherein fermentation is carried out between 20-40° C., more preferably 30-37° C.

6. The process according to any one of the preceding claims, wherein the Leuconostoc carnosum reduces hexanal in the starting material.

7. The process according to any one of the preceding claims, wherein the Leuconostoc carnosum reduces pentanal in the starting material.

8. The process according to any one of the preceding claims, wherein the Leuconostoc carnosum reduces diacetyl, benzaldehyde, furfural, octanal, nonanal, 2-hexenal, 2-heptenal and 2-octenal or 1-penten-3-ol in the starting material.

9. The process of any one of the preceding claims, wherein the Leuconostoc carnosum is Leuconostoc carnosum DSM 34220 or a mutant thereof.

10. The process according to any one of the preceding claims, wherein the composition comprises further bacteria and/or yeasts.

11. A meat analogue product obtained by the process according to any one of claims 1-10.

12. Use of a composition comprising Leuconostoc carnosum, preferably DSM 34220 or a mutant thereof, for improving the flavor of a meat analogue product prepared from plant material, preferably from legumes or cereals.

13. The use of claim 12, where in the Leuconostoc carnosum reduces the content of hexanal, pentanal, diacetyl, benzaldehyde, furfural, octanal, nonanal, 2-hexenal, 2-heptenal and 2-octenal and/or 1-penten-3-ol in the meat analogue product prepared from plant material.

14. The use of claim 12 or 13, wherein composition has a concentration of viable cells of at least 10⁴ CFU/g colony forming units (CFU)/g.

15. A meat analogue product prepared from legumes and/or cereal comprising at least 10⁵ CFU/g Leuconostoc carnosum, preferably the Leuconostoc carnosum DSM 34220 or a mutant thereof.
